(19) United States
(12) Patent Application Publication
(10) Pub. No.: US 2024/0261212 A1
(43) Pub. Date: Aug. 8, 2024

(54) Alkaline Solvent Pre-Wash of Hair For Optimizing Wash Stability of Level 1 Colorations

(71) Applicant: Henkel AG & Co. KGaA, Duesseldorf (DE)

(72) Inventors: Juergen Schoepgens, Dusseldorf (DE); Torsten Lechner, Dusseldorf (DE); Carsten Mathiaszyk, Dusseldorf (DE); Phillip Jaiser, Dusseldorf (DE); Marc Nowottny, Dusseldorf (DE); Andreas Walter, Dusseldorf (DE); Carolin Kruppa, Dusseldorf (DE); Avni Tairi, Dusseldorf (DE)

(21) Appl. No.: 18/634,387

(22) Filed: Apr. 12, 2024

Related U.S. Application Data
(63) Continuation of application No. PCT/EP2022/076685, filed on Sep. 26, 2022.

(30) Foreign Application Priority Data
Oct. 14, 2021 (DE) 102021126735.8

(51) Int. Cl. A61K 8/86; A61K 8/34; A61Q 5/00

(52) U.S. Cl. CPC A61K 8/86; A61K 8/34; A61Q 5/004; A61K 2800/5922

(57) ABSTRACT

The present application relates to a cosmetic composition for pretreatment of hair, wherein the cosmetic composition contains at least one polyethylene glycol, at least one alcohol, and at least one alkalizing agent. The present application further relates to a cosmetic method for pretreatment of hair. In addition, the present application relates to the use of an alkaline aqueous or an alkaline aqueous-alcoholic solution comprising at least one polyethylene glycol for increasing the washing stability of human hair, and to the use of the cosmetic composition for increasing the washing stability of human hair.

### FIELD OF INVENTION

The present application relates to a cosmetic composition for pretreatment of hair, wherein the cosmetic composition contains at least one polyethylene glycol, at least one alcohol, and at least one alkalizing agent. The present application further relates to a cosmetic method for pretreatment of hair. In addition, the present application relates to the use of an alkaline aqueous or an alkaline aqueous-alcoholic solution comprising at least one polyethylene glycol for increasing the washing stability of human hair, and to the use of the cosmetic composition for increasing the washing stability of human hair.

### BACKGROUND

Changing the shape and color of keratin fibers, and in particular hair, represents an important area of modern cosmetics. To change the hair color, the skilled artisan is familiar with a variety of dyeing systems depending upon the dyeing requirements. Oxidation dyes are typically used for permanent, intense dyeing with good fastness properties and good gray coverage. Such dyes typically contain oxidation dye precursors, known as developer components, and coupler components, which together form the actual dyes under the influence of oxidizing agents—for example, hydrogen peroxide. Oxidation dyes are characterized by very long-lasting color results.

When using direct dyes, dyes which are already formed diffuse out of the coloring agent into the hair fiber. In comparison with oxidative hair dyeing, the colors obtained with direct dyes have a lower durability and a more rapid washing out. Colors with direct dyes usually remain on the hair for a period of between 5 and 20 hair washes.

The use of color pigments for brief changes in color on the hair and/or the skin is known. Color pigments are generally understood to mean insoluble dyeing substances. These are present undissolved in the form of small particles in the dyeing formulation and are only deposited from the outside onto the hair fibers and/or the skin surface. They can therefore generally be removed again without leaving residue by washing a few times with surfactant-containing cleaning agents. Various products of this type by the name of hair mascara are available on the market.

If the user desires particularly long-lasting dyeing, the use of oxidative coloring agents has hitherto been the only option. However, despite multiple optimization attempts, an unpleasant ammonia odor or amine odor cannot be completely avoided in oxidative hair dyeing. The hair damage that remains associated with the use of the oxidative coloring agents also has a disadvantageous effect on the hair of the user. Therefore, many users prefer hair dyes with direct dyes and accept the low wash stability.

“Level 1 coloration,” i.e., temporary hair colorations using direct dyes which are characterized by improved washing stability, would offer a considerable technical advantage.

### SUMMARY OF THE INVENTION

The present invention provides a cosmetic composition by which improved washing stability after temporary hair dyes is achieved. In particular, the color intensity shall remain stable over the number of washing cycles of the hair after temporary hair dyes. However, it shall be possible to dispense with the use of the customarily used oxidation dye precursors, which are characterized by a very high washing stability after hair coloring. Very particular attention is given to measures that ensure improved washing stability of temporary hair dyes.

### DETAILED DESCRIPTION OF THE INVENTION

The object of the present invention is achieved by the disclosed cosmetic composition comprising, relative to the total weight of the cosmetic composition, a) 20 to 90 wt % of at least one polyethylene glycol, b) 20 to 90 wt % of at least one alcohol, and c) 0.1 to 20 wt % of at least one alkalizing agent.

The cosmetic composition achieves the above objects in that it can be or is used in pretreatment of the hair. The object is accordingly achieved in particular by the cosmetic composition for use in a pretreatment of keratin fibers, and in particular human hair, before a dyeing treatment, wherein the cosmetic composition contains a) 20 to 90 wt % of at least one polyethylene glycol, b) 20 to 90 wt % of at least one alcohol, and c) 0.1 to 20 wt % of at least one alkalizing agent.

It has been found that the presence of an 18-MEA layer (18-methyleicosanoic acid) on keratin fibers is associated with the binding capacity of components to be applied to the keratin fibers. For example, S. J. Meade et al. (published in Textile Research Journal, vol. 78(11), pages 943 to 957, 2008) found that removal of 18-methyleicosanoic acid (18-MEA) and other surface-bound lipids that cover the wool fiber surface results in the chemical and physical cleavage of thiol esters to free up functional chemical groups that are then available for the attachment of molecules to be impacted. S. J. Meade et al. use agents that are not suitable for pretreatment of human hair to remove the 18-MEA layer of animal wool fibers. In the prior art, they use methanolic t-butoxide solutions or physical processes such as plasma treatments. Beyond the teachings of S. J. Meade et al., measures have been found in the application described here with which the washing stability can be improved after temporary colorations without using cosmetically unacceptable agents.

It was found that the cosmetic compositions described here are suitable for carrying out a pretreatment by which the washing stability can be increased: non-pretreated hair strands on which a direct coloring (level 1) was applied show significantly restricted washing stabilities. Even after just a few hair washes, a majority of the coloring components of the temporary hair dyes which have attached to the surface of the hair are lost, and the color intensity decreases thereby. Applied pretreatment is different: Pretreatment of keratin fibers, and especially human hair, creates a fiber or hair surface to which molecules, and especially direct dyes, appear to bind more strongly. As a result, the pretreatment of the hair achieves a higher washing stability of the coloring agents on the hair after temporary hair coloration.

As a first component, the cosmetic composition contains one or more polyethylene oxides (polyethylene glycols). Polyethylene oxide is to be understood as meaning a polymer composed of at least three ethylene oxide units. According to a preferred embodiment, the cosmetic composition contains polyethylene glycol which has a weight-averaged molecular weight of 200 to 9,000 g/mol, preferably 250 to 5,000 g/mol, more preferably 300 to 2,500 g/mol, and most preferably 400 to 1,000 g/mol. The weight-averaged molecular weight (Mw) is measured with gel permeation chromatography (GPC), and preferably with polyethylene oxide as the standard.

It is furthermore preferred that two or more polyethylene glycols different from one another be included as the polyethylene glycol in the cosmetic composition, which differ by their average weight-averaged molecular weight. Two or more different polyethylene glycols are mixtures which have a bimodal or multimodal distribution of the Mw. In this way, it can be achieved that the mixture of the two or more polyethylene glycols remains fluid when the mixture is considered in isolation, although a higher molecular weight polyethylene glycol is present which is viscous or solid.

Various polyethylene glycols are commercially available. For the present cosmetic composition, the following are preferably used: PEG 300, PEG 400, PEG 600, PEG 1500, PEG 3000, PEG 4000, PEG 6000, and PEG 8000. These can be used individually or as a mixture of two or more thereof in the cosmetic composition.

According to the present application, the amount of contained polyethylene glycol is 20 to 90 wt %, preferably 30 to 80 wt %, more preferably 40 to 70 wt %, and most preferably 45 to 60 wt % in the cosmetic composition. If the polyethylene glycol is a special polyethylene glycol or a special mixture of special polyethylene glycols, then preferably the total amount of all polyethylene glycols as a generic term is within the stated amount of the generic term.

As a second component, the cosmetic composition contains one or more alcohols. According to a preferred embodiment, the alcohol is a primary or secondary alcohol, or the alcohol is a monoalkanol, preferably a C₁₋₁₂ monoalcohol, more preferably a C₁₋₆ monoalcohol, and more preferably ethanol, propanol, isopropanol, n-butanol, or tert-butanol. According to a very preferred embodiment, the alcohol is ethanol. By definition, the alcohol should be free of a basic group.

The amount of alcohol contained in the cosmetic composition is 20 to 90 wt %, preferably 30 to 80 wt %, more preferably 35 to 75 wt %, and most preferably 40 to 70 wt %. If the alcohol is a special alcohol or a specific mixture of special alcohols, the total amount of all alcohols as a generic term is preferably within the stated amount of the generic term.

As a third component, the cosmetic composition contains an alkalizing agent. According to a preferred embodiment, the alkalizing agent is selected from the group consisting of ammonium hydroxide, ammonia, a basic amino acid, an alkali metal hydroxide, and in particular sodium hydroxide or potassium hydroxide, an alkanolamine, and in particular aminoethanol, an alkali metal metasilicate, an alkali phosphate and an alkali hydrogen phosphate, and mixtures thereof. The alkalizing agent ensures the setting of the suitable pH value.

By definition, an alkalizing agent that has an OH group should not be included among the alcohols. The alcohol and the alkalizing agent differ from one another. An alcohol according to the present application is not an alkalizing agent, and an alkalizing agent of the present application is not an alcohol.

The amount of alkalizing agent contained in the cosmetic composition is 0.1 to 20 wt %, preferably 0.2 to 10 wt %, more preferably 0.5 to 8 wt %, even more preferably 1 to 6 wt %, and most preferably 2 to 4 wt %. If the alkalizing agent is a special alkalizing agent or a special mixture of special alkalizing agents, then preferably the total amount of all alkalizing agents as a generic term is within the stated amount of the generic term.

All percentages by weight are always based upon the cosmetic composition, unless expressly disclosed otherwise. Of course, the percentages by weight of all components in the cosmetic composition always add up to 100 wt %.

Preferred amounts (percentages by weight) of the respective components are further derived from the statements below.

According to a further preferred embodiment, the cosmetic composition substantially consists of the at least one polyethylene glycol, the at least one alcohol, the alkalizing agent, and water. The term, “substantially,” means that the at least one polyethylene glycol, the at least one alcohol, the alkalizing agent, and water are present in total in an amount, relative to the total weight of the cosmetic composition, of 95 wt % and more, preferably 98 wt % and more, and more preferably 99 wt %, wherein the remainder consists of excipients which can be selected or are selected from perfumes, preservatives, vitamins, and care substances.

The present invention was furthermore based upon the object of providing a method which enables or brings about the increase in washing stability after temporary hair coloration.

The object is achieved by a cosmetic method for pretreatment of keratin fibers, and in particular human hair, before a dyeing treatment of the keratin fibers, and in particular of human hair, in which:
a cosmetic composition according to the present description is applied to the keratin fibers, and in particular to human hair,
the cosmetic composition is allowed to act for a time of 5 seconds to 60 minutes, preferably 10 seconds to 15 minutes, more preferably 30 seconds to 7 minutes, and most preferably 1 minute to 4 minutes, and
the keratin fibers, and in particular human hair, are rinsed.

The method according to this description comprises the application of a cosmetic composition according to the present description to the hair. The cosmetic composition is allowed to act for a period of time which is sufficient to remove the 18-MEA layer at least partially from the surface of the keratin fiber, and in particular from human hair. The keratin fibers, and in particular human hair, are then rinsed. If necessary, the hair can be dried. However, it is preferred that the coloring treatment be performed immediately after the cosmetic composition is rinsed. The dyeing treatment follows the pretreatment without drying the hair.

The method described here allows intermediate steps to be carried out. For example, the cosmetic composition described herein is massaged in with the hands after or during application. Furthermore, the massaging can be carried out while the cosmetic composition is allowed to act. During or between the process steps, a heat treatment step may be applied. The method preferably ends at the beginning of the dyeing treatment. The dyeing treatment is in particular dyeing with direct dyes.

The at least partially removed 18-MEA layer enables a stronger bonding of dyes on the hair.

The time between the start of the rinsing step and the end of the cosmetic method should be short. After the rinsing of the cosmetic composition and the start of the dying step, there is preferably a time of about 1 second to 10 hours, and more preferably from 30 seconds to 1 hour.

In addition, a method is hereby disclosed which comprises the cosmetic method described herein for pretreatment of keratin fibers, and in particular human hair, and coloring with temporary coloring agents. The method of pretreatment described above is followed by the step of dyeing the keratin fibers, and in particular human hair, with a temporary coloring agent. In other words, the fourth step of this preferred method is level 1 coloration.

The following coloring agents can be used in the context of this disclosure. The coloring agents/dyes are used according to the pretreatment method according to this disclosure:

The coloring agent contains at least one anionic, cationic, and/or non-ionic direct dye.

Suitable cationic direct dyes are, for example, Basic Blue 7, Basic Blue 26, Basic Violet 2 and Basic Violet 14, Basic Yellow 57, Basic Red 76, Basic Blue 16, Basic Blue 347 (Cationic Blue 347/Dystar), HC Blue No. 16, Basic Blue 99, Basic Brown 16, Basic Brown 17, Basic Yellow 57, Basic Yellow 87, Basic Orange 31, Basic Red 51, Basic Red 76.

Non-ionic nitro dyes and quinone dyes and neutral azo dyes, for example, can be used as non-ionic direct dyes. Suitable non-ionic direct dyes are the compounds known under the international names or trade names, HC Yellow 2, HC Yellow 4, HC Yellow 5, HC Yellow 6, HC Yellow 12, HC Orange 1, Disperse Orange 3, HC Red 1, HC Red 3, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9, and also 1,4-diamino-2-nitrobenzene, 2-amino-4-nitrophenol, 1,4-bis (2-hydroxyethyl)amino-2-nitrobenzene, 3-nitro-4-(2-hydroxy-ethyl)aminophenol, 2-(2-hydroxyethyl)amino-4,6-dinitrophenol, 4-[(2-hydroxyethyl)amino]-3-nitro-1-methylbenzene, 1-amino-4-(2-hydroxyethyl)amino-5-chloro-2-nitrobenzene, 4-amino-3-nitrophenol, 1-(2′-ureidoethyl)amino-4-nitrobenzene, 2-[(4-amino-2-nitrophenyl)amino]benzoic acid, 6-nitro-1,2,3,4-tetrahydroquinoxaline, 2-hydroxy-1,4-naphthoquinone, picramic acid and salts thereof, 2-amino-6-chloro-4-nitrophenol, 4-ethylamino-3-nitrobenzoic acid, and 2-chloro-6-ethylamino-4-nitrophenol.

A suitable coloring agent comprises at least one anionic direct dye. Anionic direct dyes are also referred to as acid dyes. Acid dyes mean direct dyes that have at least one carboxylic acid group (—COOH) and/or a sulfonic acid group (—SO₃H). Depending upon the pH, the protonated forms (—COOH, —SO₃H) of carboxylic acid or sulfonic acid groupings are present in equilibrium with their deprotonated forms (—COO—, —SO₃—). The proportion of the protonated forms increases with a decreasing pH. If direct dyes are used in the form of their salts, the carboxylic acid groups or sulfonic acid groups are present in the deprotonated form and are neutralized with corresponding stoichiometric equivalents of cations in order to maintain electroneutrality.

Suitable chromophore systems are found, for example, in the structures of nitrophenylenediamines, nitroaminophenols, azo dyes, anthraquinone dyes, triarylmethane dyes, xanthene dyes, rhodamine dyes, oxazine dyes, and/or indophenol dyes.

For example, as particularly well-suited acid dyes, one or more compounds can be selected from the following group: Acid Yellow 1 (D&C Yellow 7, Citronin A, Ext. D&C Yellow No. 7, Japan Yellow 403, CI 10316, COLIPA n° B001), Acid Yellow 3 (COLIPA n°: C 54, D&C Yellow N° 10, Quinoline Yellow, E104, Food Yellow 13), Acid Yellow 9 (CI 13015), Acid Yellow 17 (CI 18965), Acid Yellow 23 (COLIPA n° C. 29, Covacap Jaune W 1100 (LCW), Sicovit Tartrazine 85 E 102 (BASF), Tartrazine, Food Yellow 4, Japan Yellow 4, FD&C Yellow No. 5), Acid Yellow 36 (CI 13065), Acid Yellow 121 (CI 18690), Acid Orange 6 (CI 14270), Acid Orange 7 (2-Naphthol orange, Orange II, CI 15510, D&C Orange 4, COLIPA n° C.015), Acid Orange 10 (CI 16230; Orange G sodium salt), Acid Orange 11 (CI 45370), Acid Orange 15 (CI 50120), Acid Orange 20 (CI 14600), Acid Orange 24 (BROWN 1; CI 20170; KATSU201; no sodium salt; Brown No. 201; RESORCIN BROWN; ACID ORANGE 24; Japan Brown 201; D & C Brown No. 1), Acid Red 14 (CI14720), Acid Red 18 (E124, Red 18; CI 16255), Acid Red 27 (E 123, CI 16185, C-Rot 46, Echtrot D, FD&C Red No. 2, Food Red 9, Naphtholrot S), Acid Red 33 (Red 33, Fuchsia Red, D&C Red 33, CI 17200), Acid Red 35 (CI C.I.18065), Acid Red 51 (CI 45430, Pyrosin B, Tetraiodfluorescein, Eosin J, Iodeosin), Acid Red 52 (CI 45100, Food Red 106, Solar Rhodamine B, Acid Rhodamine B, Red n° 106 Pontacyl Brilliant Pink), Acid Red 73 (CI CI 27290), Acid Red 87 (Eosin, CI 45380), Acid Red 92 (COLIPA n° C.53, CI 45410), Acid Red 95 (CI 45425, Erythtosine, Simacid Erythrosine Y), Acid Red 184 (CI 15685), Acid Red 195, Acid Violet 43 (Jarocol Violet 43, Ext. D&C Violet no. 2, CI 60730, COLIPA n° C.063), Acid Violet 49 (CI 42640), Acid Violet 50 (CI 50325), Acid Blue 1 (Patent Blue, CI 42045), Acid Blue 3 (Patent Blue V, CI 42051), Acid Blue 7 (CI 42080), Acid Blue 104 (CI 42735), Acid Blue 9 (E 133, Patentblau AE, Amidoblau AE, Erioglaucin A, CI 42090, CI Food Blue 2), Acid Blue 62 (CI 62045), Acid Blue 74 (E 132, CI 73015), Acid Blue 80 (CI 61585), Acid Green 3 (CI 42085, Foodgreen1), Acid Green 5 (CI 42095), Acid Green 9 (CI 42100), Acid Green 22 (CI 42170), Acid Green 25 (CI 61570, Japan Green 201, D&C Green No. 5), Acid Green 50 (Brilliant Acid Green BS, CI 44090, Acid Brilliant Green BS, E 142), Acid Black 1 (Black no. 401, Naphthalene Black 10B, Amido Black 10B, CI 20 470, COLIPA no. B15), Acid Black 52 (CI 15711), Food Yellow 8 (CI 14270), Food Blue 5, D&C Yellow 8, D&C Green 5, D&C Orange 10, D&C Orange 11, D&C Red 21, D&C Red 27, D&C Red 33, D&C Violet 2, and/or D&C Brown 1.

In addition, the object upon which the invention is based is to provide a measure which increases the washing stability after temporary hair coloration.

The object is achieved by the use of an alkaline aqueous or an alkaline aqueous-alcoholic solution comprising at least one polyethylene glycol for increasing the washing stability of keratin fibers, and in particular human hair, which comprises the use of the alkaline aqueous or alkaline aqueous-alcoholic solution before a dyeing treatment of the keratin fibers, and in particular of human hair.

Surprisingly, it has been found that polyethylene glycol in an alkaline aqueous or preferably an alkaline aqueous-alcoholic solution is capable of changing the hair surface in such a way that dyes bind more strongly to the hair surface. The use of the solution in a method for pretreatment of hair increases the washing stability after the dyeing treatment.

Furthermore, the object is achieved by the use of a cosmetic composition according to the above description for increasing the washing stability of keratin fibers, and in particular human hair, which comprises the use of the cosmetic composition before a dyeing treatment of the keratin fibers, and in particular of human hair.

According to the present description, the application of the cosmetic composition described herein allows the at least partial removal of the 18-MEA layer and/or further surface-bound lipids, so that the temporary dyeing treatment, which follows the pretreatment with the cosmetic composition described here, yields a coloring result that is characterized by increased washing stability.

According to a preferred embodiment, the dyeing treatment is one with a direct dye.

Of course, features which are disclosed only in connection with the cosmetic composition described here can serve as features for the method described here or for the uses described here.

The invention can also be defined by the following statements:

1. A cosmetic composition comprising, relative to the total weight of the cosmetic composition,
a. 20 to 90 wt % of at least one polyethylene glycol,
b. 20 to 90 wt % of at least one alcohol, and
c. 0.1 to 20 wt % of at least one alkalizing agent.

2. A cosmetic composition comprising, relative to the total weight of the cosmetic composition,
a. 30 to 80 wt % of at least one polyethylene glycol,
b. 30 to 80 wt % of at least one alcohol, and
c. 0.1 to 10 wt % of at least one alkalizing agent.

3. A cosmetic composition comprising, relative to the total weight of the cosmetic composition,
a. 40 to 70 wt % of at least one polyethylene glycol,
b. 35 to 70 wt % of at least one alcohol, and
c. 0.2 to 8 wt % of at least one alkalizing agent.

4. A cosmetic composition comprising, relative to the total weight of the cosmetic composition,
a. 45 to 70 wt % of at least one polyethylene glycol,
b. 35 to 65 wt % of at least one alcohol, and
c. 0.5 to 6 wt % of at least one alkalizing agent.

5. A cosmetic composition comprising, relative to the total weight of the cosmetic composition,
a. 45 to 60 wt % of at least one polyethylene glycol,
b. 35 to 60 wt % of at least one alcohol, and
c. 1 to 4 wt % of at least one alkalizing agent.

6. A cosmetic composition comprising, relative to the total weight of the cosmetic composition,
a. 45 to 55 wt % of at least one polyethylene glycol,
b. 40 to 55 wt % of at least one alcohol, and
c. 2 to 3 wt % of at least one alkalizing agent.

7. The cosmetic composition according to one of the preceding statements, characterized in that the amount of polyethylene glycol is greater than the amounts of alcohol.

8. The cosmetic composition according to one of the preceding statements, characterized in that the polyethylene glycol has a weight-averaged molecular weight of 200 to 9,000 g/mol, preferably 250 to 5,000 g/mol, more preferably 300 to 2,500 g/mol, and most preferably 400 to 1,000 g/mol.

9. The cosmetic composition according to one of the preceding statements, characterized in that two or more polyethylene glycols different from one another are included as polyethylene glycol in the cosmetic composition and differ by their average weight-averaged molecular weight.

10. The cosmetic composition according to one of the preceding statements, characterized in that one or two or more polyethylene glycols selected from the group consisting of PEG 300, PEG 400, PEG 600, PEG 1500, PEG 3000, PEG 4000, PEG 6000, and PEG 8000 are used as the polyethylene glycol.

11. The cosmetic composition according to one of the preceding statements, characterized in that two polyethylene glycols selected from the group consisting of PEG 300, PEG 400, PEG 600, PEG 1500, PEG 3000, PEG 4000, PEG 6000, and PEG 8000 are used as the polyethylene glycol.

12. The cosmetic composition according to one of the preceding statements, characterized in that the one or more polyethylene glycols, as used for the cosmetic composition, is or are liquid under normal conditions (standard conditions according to IUPAC).

13. The cosmetic composition according to one of the preceding statements, characterized in that the alcohol is a primary alcohol, secondary alcohol, or a monoalkanol.

14. The cosmetic composition according to one of the preceding statements, characterized in that the alcohol is a C₁₋₁₂ monoalcohol, and preferably a C₁₋₆ monoalcohol.

15. The cosmetic composition according to one of the preceding statements, characterized in that the alcohol is selected from the group consisting of ethanol, propanol, isopropanol, n-butanol, and tert-butanol, and in particular ethanol.

16. The cosmetic composition according to one of the preceding statements, characterized in that the alkalizing agent is selected from the group consisting of ammonium hydroxide, ammonia, a basic amino acid, an alkali metal hydroxide, and in particular sodium hydroxide or potassium hydroxide, an alkanolamine, and in particular aminoethanol, an alkali metal metasilicate, an alkali phosphate, and an alkali hydrogen phosphate, and mixtures thereof.

17. The cosmetic composition according to one of the preceding statements, characterized in that the cosmetic composition, relative to the total weight of the cosmetic composition,
d) contains 1 to 30 wt %, preferably 2 to 20 wt %, preferably 3 to 15 wt %, and particularly 4 to 10 wt % water.

18. The cosmetic composition according to one of the preceding statements, characterized in that the alkalizing agent in the cosmetic composition is contained in an amount such that the pH value would be from 8 to 16, preferably 10 to 15.5, more preferably 11 to 15, and most preferably 12 to 14, if the alkalizing agent were contained isolated in the amount of water contained in the cosmetic composition.

19. The cosmetic composition according to one of the preceding statements, characterized in that the cosmetic composition consists substantially of the at least one polyethylene glycol, the at least one alcohol, the alkalizing agent, and water.

20. The cosmetic composition according to one of the preceding statements, characterized in that the cosmetic composition comprises from 0 to 5 wt %, preferably 0.1 to 2 wt %, and more preferably 0.5 to 2 wt %, of one or more excipients.

21. The cosmetic composition according to statement 20, characterized in that the excipient is selected from the group consisting of perfumes, preservatives, vitamins, care substances, and combinations thereof.

22. The cosmetic composition according to one of the preceding statements, characterized in that the sum of the amount of the at least one polyethylene glycol and the at least one alcohol is between 60 and 95 wt %, preferably between 70 and 90 wt %, and more preferably between 75 and 85 wt %.

23. The cosmetic composition according to one of the preceding statements, characterized in that the cosmetic composition is free of organosilicon compounds.

24. The cosmetic composition according to one of the preceding statements, characterized in that the cosmetic composition is free of peroxides.

25. The cosmetic composition according to one of the preceding statements, characterized in that the cosmetic composition is used to increase the washing stability.

26. The cosmetic composition according to one of the preceding statements, characterized in that the cosmetic composition is used to increase the washing stability after a dyeing treatment with a direct dye.

27. A cosmetic method for pretreatment of keratin fibers, and in particular human hair, before a dyeing treatment of the keratin fibers, and in particular of human hair, in which a cosmetic composition according to one of statements 1 through 26 is applied to the keratin fibers, and in particular to human hair, the cosmetic composition is allowed to act for a time of 5 seconds to 60 minutes, preferably 10 seconds to 15 minutes, more preferably 30 seconds to 7 minutes, and most preferably 1 minute to 4 minutes, and the keratin fibers, and in particular human hair, are rinsed.

28. A use of an alkaline aqueous or an alkaline aqueous-alcoholic solution comprising at least one polyethylene glycol for increasing the washing stability of keratin fibers, and in particular human hair, which comprises the use of the alkaline aqueous or the alkaline aqueous-alcoholic solution before a dyeing treatment of the keratin fibers, and in particular of human hair.

29. The use according to statement 28, characterized in that the polyethylene glycol has a weight-averaged molecular weight of 200 to 9,000 g/mol, preferably 250 to 5,000 g/mol, more preferably 300 to 2,500 g/mol, and most preferably 400 to 1,000 g/mol.

30. The use according to statement 28 or statement 29, characterized in that two or more different polyethylene glycols are contained as the polyethylene glycol in the cosmetic composition, which differ by their average weight-averaged molecular weight.

31. The use according to one of statements 28 through 30, characterized in that, as the polyethylene glycol, one or two or more polyethylene glycols are used which are selected from the group consisting of PEG 300, PEG 400, PEG 600, PEG 1500, PEG 3000, PEG 4000, PEG 6000, and PEG 8000.

32. The use according to one of statements 28 through 31, characterized in that, as the polyethylene glycol, two polyethylene glycols are used which are selected from the group consisting of PEG 300, PEG 400, PEG 600, PEG 1500, PEG 3000, PEG 4000, PEG 6000, and PEG 8000.

33. The use according to one of statements 28 through 32, characterized in that the one or the two or more polyethylene glycols, as used for the cosmetic composition, is or are liquid under normal conditions (standard conditions according to IUPAC).

34. The use of a cosmetic composition according to one of claims 1 through 26 for increasing the washing stability of keratin fibers, and in particular human hair, which comprises the use of the cosmetic composition before a dyeing treatment of the keratin fibers, and in particular of human hair.

35. The use according to one of statements 28 through 34, characterized in that the dyeing treatment is a direct dye.

Of course, features relating to statements that are disclosed only in connection with the cosmetic composition can also serve as features of the method or the uses.

### Example

| | Wt % |
|---|---|
| Polyethylene glycol MG 400 | 50 |
| Ethanol 99% denat. (t-butanol, Bitrex) | 40 |
| Potassium hydroxide 50% | 2.8 |
| Water, demineralized | 7.2 |
| Total | 100 |

The components according to the above table are combined and mixed. The resulting mixture is applied to the hair and massaged for about 2 min. The mixture is then rinsed out with water. Immediately thereafter, a level 1 coloration is applied.

What is claimed is:

1. A cosmetic composition comprising, relative to the total weight of the cosmetic composition,
a. 20 to 90 wt % of at least one polyethylene glycol,
b. 20 to 90 wt % of at least one alcohol, and
c. 0.1 to 20 wt % of at least one alkalizing agent.

2. The cosmetic composition according to claim 1, wherein the at least one polyethylene glycol has a weight-averaged molecular weight of 200 to 9,000 g/mol.

3. The cosmetic composition according to claim 1, wherein the at least one polyethylene glycol has a weight-averaged molecular weight of 250 to 5,000 g/mol.

4. The cosmetic composition according to claim 1, wherein the at least one polyethylene glycol has a weight-averaged molecular weight of 300 to 2,500 g/mol.

5. The cosmetic composition according to claim 1, wherein the at least one polyethylene glycol has a weight-averaged molecular weight of 400 to 1,000 g/mol.

6. The cosmetic composition according to claim 1, wherein the at least one polyethylene glycol is selected from the group consisting of PEG 300, PEG 400, PEG 600, PEG 1500, PEG 3000, PEG 4000, PEG 6000, and PEG 8000.

7. The cosmetic composition according to claim 1, wherein at least one polethylene glyocols includes two or more different polyethylene glycols which differ by their average weight-averaged molecular weight.

8. The cosmetic composition according to claim 1, wherein the at least one alcohol is a primary alcohol, secondary alcohol, a monoalkanol, or mixtures thereof.

9. The cosmetic composition according to claim 1, wherein the at least one alcohol is a C₁₋₁₂ monoalcohol.

10. The cosmetic composition according to claim 1, wherein the at least one alcohol is a C₁₋₆ monoalcohol.

11. The cosmetic composition according to claim 1, wherein the at least one alcohol is selected from the group consisting of ethanol, propanol, isopropanol, n-butanol, or tert-butanol, and mixtures thereof.

12. The cosmetic composition according to claim 1, wherein the at least one alkalizing agent is selected from the group consisting of ammonium hydroxide, ammonia, a basic amino acid, an alkali metal hydroxide, an alkanolamine, an alkali metal metasilicate, an alkali phosphate, and an alkali hydrogen phosphate, and mixtures thereof.

13. The cosmetic composition according to claim 1, wherein the at least one alkalizing agent is selected from the group consisting of sodium hydroxide, potassium hydroxide, and aminoethanol.

14. The cosmetic composition according to claim 1, wherein the cosmetic composition consists substantially of the at least one polyethylene glycol, the at least one alcohol, the alkalizing agent, and water.

15. A cosmetic method for pretreatment of keratin fibers, before a dyeing treatment of the keratin fibers, comprising:
applying a cosmetic composition according to claim 1 to the keratin fibers,
leaving the cosmetic composition on the keratin fibers for a period of time of 5 seconds to 60 minutes,
rinsing the keratin fibers.

16. The method according to claim 15, wherein the composition is left on the keratin fibers for a period of 10 seconds to 15 minutes.

17. The method according to claim 15, wherein the composition is left on the keratin fibers for a period of 30 seconds to 7 minutes.

18. The method according to claim 15, further comprising dying the keratin fibers with a direct coloring agent.
